(19) United States
(12) Patent Application Publication
(10) Pub. No.: US 2025/0366983 A1
(43) Pub. Date: Dec. 4, 2025

(54) INTRAOCULAR LENS WITH METASURFACE ELEMENTS FOR REDUCING REFLECTIONS

(71) Applicant: Alcon Inc., Fribourg (CH)

(72) Inventors: Behzad BORDBAR, Fort Worth, TX (US); Austin RODEHEAVER, Fort Worth, TX (US); Xin HONG, Fort Worth, TX (US); Shinwook LEE, Arlington, TX (US); Daniel CARSON, Fort Worth, TX (US); Stephen VAN NOY, Fort Worth, TX (US); Marcia ONG, Fort Worth, TX (US); Shridhar NATARAJAN, Fort Worth, TX (US); Amjad CHATILA, Fort Worth, TX (US)

(21) Appl. No.: 19/220,084

(22) Filed: May 28, 2025

Related U.S. Application Data
(60) Provisional application No. 63/652,577, filed on May 28, 2024.

(51) Int. Cl. A61F 2/16; G02C 7/06

(52) U.S. Cl. CPC A61F 2/1654; A61F 2/1618; G02C 7/06; A61F 2002/169; G02C 2202/20

(57) ABSTRACT

An IOL includes an optic having an anterior optic surface configured to receive light passing into an eye in which the IOL is implanted and a posterior optic surface opposite the anterior surface. The IOL includes at least one of refractive and diffractive structures configured to improve vision of the eye. A metasurface is configured to reduce reflection in the human visible spectrum from the anterior surface. The IOL further may further include a peripheral portion surrounding the optic, a metasurface may additionally or alternatively be formed on a peripheral portion and be configured to act as a filter to reduce transmission in the human visible spectrum into the peripheral portion through a peripheral anterior surface of the peripheral portion.

### CROSS-REFERENCE TO RELATED APPLICATIONS

This application claim benefit of and priority to U.S. Provisional Patent Application No. 63/652,577, filed May 28, 2024, which is hereby assigned to the assignee hereof and hereby expressly incorporated by reference in its entirety as if fully set forth below and for all applicable purposes.

### BACKGROUND

The human eye provides vision by transmitting light through a clear outer portion called the cornea and focusing the image onto a retina via a lens. The quality of the focused image depends on many factors including the size and shape of the eye, and the transparency of the cornea and lens. As age or disease causes the lens to become opaque, vision deteriorates because of the diminished light that can be transmitted to the retina. This deficiency in the lens of the eye is medically known as a cataract. One treatment for this condition is to surgically remove the lens and implant an intraocular lenses (IOL).

Although existing IOLs may be acceptable, they also have certain shortcomings. Accordingly, there is a need for improvements to IOL designs.

### SUMMARY

In one aspect of the invention, an IOL includes an optic having an anterior optic surface configured to receive light passing into an eye in which the IOL is implanted and a posterior optic surface opposite the anterior surface. The IOL includes at least one of refractive and diffractive structures configured to improve vision of the eye. A metasurface is configured to reduce reflection in the visible spectrum from the anterior surface.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is noted, however, that the appended drawings illustrate only some aspects of this disclosure and the disclosure may admit to other equally effective embodiments.

FIG. 1 illustrates a conventional intraocular lens (IOL) and an eye.

FIG. 2 is an isometric view of an IOL showing a cross-section thereof in accordance with certain embodiments.

FIG. 3 is a partial cross-section view of the IOL of FIG. 2 showing behavior of near-normal rays in accordance with certain embodiments.

FIG. 4A is a cross-section view of an anterior surface region of the IOL having a metasurface formed thereon in accordance with certain embodiments.

FIG. 4B is a cross-section view of an anterior surface region of the IOL having an alternative implementation of a metasurface formed thereon in accordance with certain embodiments.

FIGS. 5A to 5C are cross-section views of pillars of a metasurface in accordance with certain embodiments.

FIG. 6 is a partial cross-section view of the IOL of FIG. 2 showing behavior of rays incident on an upper surface of a periphery of the IOL in accordance with certain embodiments.

FIG. 7 is a partial cross-section view of the IOL of FIG. 2 showing behavior of rays within a periphery of the IOL that are incident on an edge surface of the IOL in accordance with certain embodiments.

FIG. 8 is a partial cross-section view of the IOL of FIG. 2 showing behavior of rays exiting a lower surface of a periphery of the IOL in accordance with certain embodiments.

FIG. 9 is a cross-section view of a posterior surface region of the IOL having a metasurface formed thereon in accordance with certain embodiments.

FIG. 10 depicts an example method for forming a metasurface of an IOL.

FIG. 11 depicts an example system for designing, configuring, and/or forming an IOL, according to certain embodiments.

FIG. 12 depicts example operations for forming an IOL, according to certain embodiments.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

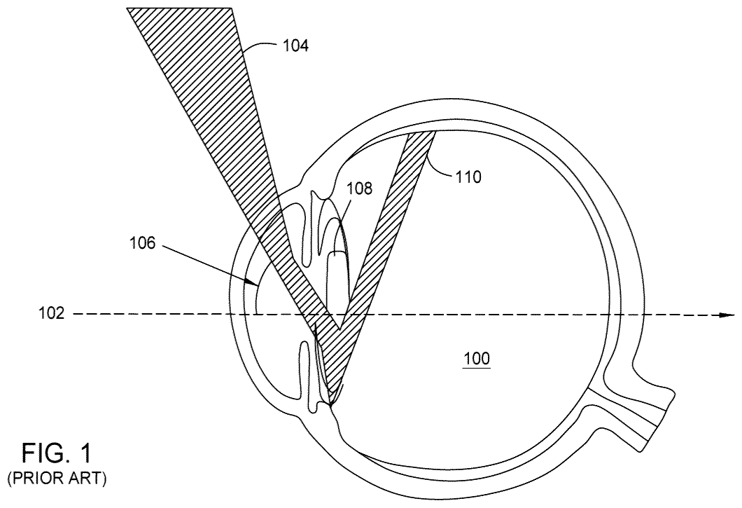

FIG. 1 illustrates a conventional intraocular lens (IOL) 108, with an optical axis 102, which is arranged within an eye 100. Although existing IOLs may function acceptably well in many patients, they also have certain shortcomings. In some cases, flashes on a vision periphery may occur after cataract surgery due to shadows and reflections related to the IOL 108.

For example, incident light rays 104 may enter the IOL 108 from a temporal direction at extreme incident angles such as incident angle 106. In such cases, a reflected light ray 110 may be perceived as a flash or reflected glare image (e.g., positive dysphotopsia or “PD”).

An innovative insight is that the geometrical characteristics (e.g., edge design) of the IOL 108 differs from the human lens that the IOL 108 replaces, with edge and/or periphery design of an IOL playing a significant role in causing and therefore potentially reducing positive dysphotopsia, as may be described in the below embodiments.

Another shortcoming of existing IOLs 108 is reflectance from the anterior surface of the IOL 108. Because of the material properties of the IOL differ from the surrounding fluid (the aqueous humor), the pupils of the patient may emit light in a way that is perceptible and disconcerting to others, a phenomenon colloquially referred to as “crazy eye.”

The embodiments described herein provide an IOL with metasurface elements (e.g., pillars) arranged on or in the IOL for, among other possibilities, reducing one or both of crazy eye and positive dysphotopsia. Metasurfaces include nanostructures (e.g., pillars or other periodic structures) that, via various designs and arrangements, may impart customized polarization, wavelength dependent amplitude (e.g., optical filtering), and/or phase to incident light and/or provide customized reflectivity of incident light.

As used herein, “metasurface” and “metasurface elements” include metasurfaces that (1) define (or partially define) an external anterior or posterior surface of a transparent member, (2) embedded metasurfaces that reside within a transparent member, and/or (3) both. In some embodiments, metasurfaces may include structures or features that are of a microscopic or nanoscopic size. For example, as is further described within this disclosure, metasurfaces may include structures on a nanoscale level, or nanostructures, such as nanopillars or nanotapers. In some aspects, the metasurface elements are arranged to be at least substantially opaque to and/or reduce reflectivity of at least a portion of the human visible spectrum for some range of angles of incidence. As used herein, “the human visible spectrum” may be defined as from 380 nm to 750 nm.

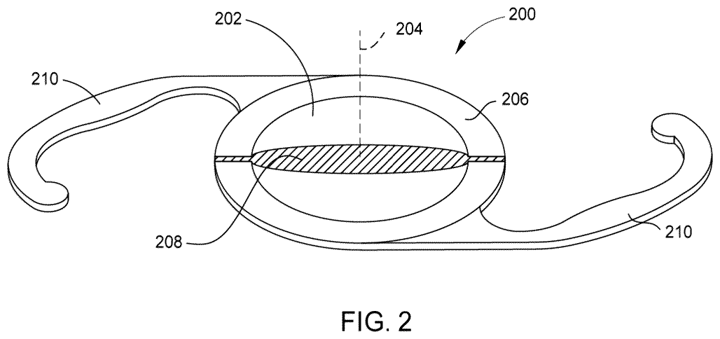

Referring to FIG. 2, an intraocular lens (IOL) 200, according to certain embodiments, includes an optic 202. The optic 202 may be embodied as a monofocal refractive lens or a multi- focal lens including refractive and diffractive structures (e.g., annular echellettes) providing multiple focal lengths, such as for some or all of distance, intermediate, and near vision. The diffractive structures may be formed on one or both of an anterior surface (facing outwardly from the eye) or posterior surface (facing the retina) of the optic 202. The optic 202 defines an optical axis 204 passing through the center of the optic 202. The focusing properties of the optic 202 may be designed for an angular region about the optical axis 204 which will be aligned with the highest resolution portion of the retina, the fovea, during use. The optic 202 may have diameter about the optical axis 204 of between about 4.5 millimeter (mm) and about 7.5 mm.

The intraocular lens 200 may include a peripheral portion 206 extending around the optic 202. For example, the peripheral portion 206 may have a toroidal shape that is thinner parallel to the optical axis 204 than a width thereof perpendicular to the optical axis 204. The peripheral portion 206 may have a rounded or square edge. For example, the optic 202 and peripheral portion 206 may have the illustrated cross-sectional shape 208.

In IOL 200, haptics 210 are mounted to the peripheral portion 206 and extend outwardly therefrom. The haptics 210 function as springs that are biased outwardly against the capsular bag of the eye in order to stabilize and maintain the IOL 200 in position. An IOL 200 according to the embodiments disclosed herein may include any type of haptics 210 known in the art and, in certain embodiments, may also lack haptics 210. In certain embodiments, IOL 200 may be implemented as a toric IOL, and may therefore have asymmetric properties about the optical axis 204 in order to compensate for astigmatism. In certain other embodiments, IOL 200 may be a non-toric, monofocal, multifocal, or any other type of IOL.

The optic 202, peripheral portion 206, and possibly the haptics 210, may be fabricated from a transparent, flexible, biocompatible polymer, such as flexible polymer, including hydrophobic acrylic polymeric materials. In some embodiments, the optic 202, the peripheral portion 206, and the haptics 210 may be made of substantially the same material, while in other embodiments, the haptics 210 may be made of a distinct material from the optic 202 and peripheral portion 206 and secured to the peripheral portion by welds, adhesive, or another fastener. For example, in some embodiments, the optic 202, the peripheral portion 206, and/or the haptics 210 may include a biocompatible material, such as modified polymethyl methacrylate (PMMA), modified PMMA hydrogels, hydroxyethyl methacrylate (HEMA), PVA hydrogels, other silicone polymeric materials, hydrophobic acrylic polymeric materials, for example, AcrySof® and Clareon® materials, available from Alcon, Inc., Fort Worth, Texas.

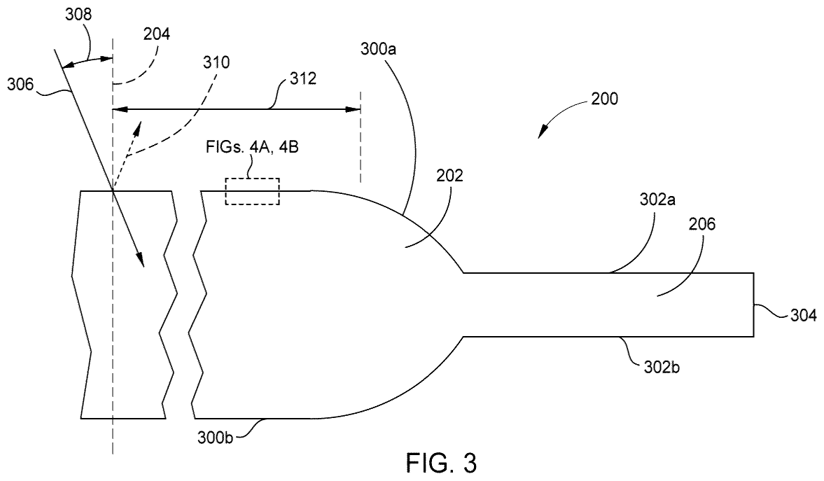

Referring to FIG. 3, the optic 202 has an anterior surface 300a and a posterior surface 300b on opposite sides of the optic 202 along the optical axis 204. The peripheral portion 206 likewise has an anterior surface 302a and a posterior surface 302b on opposite sides of the peripheral portion 206 along the optical axis 204. In the illustrated embodiment, the anterior and posterior surfaces 302a, 302b of the peripheral portion 206 are planar, with the optical axis 204 being normal to the anterior and posterior surfaces 302a, 302b. However, other shapes are possible, including conical, elliptical, or the like. An edge surface 304 extends between the anterior surface 302a and the posterior surface 302b of the peripheral portion 206. The edge surface 304 may have a cylindrical shape centered on the optical axis 204 with possible non-cylindrical features in regions secured to the haptics 210. The edge surface 304 may also have a rounded shape in planes parallel to the optical axis 204. In some embodiments, a square-edge of the peripheral portion 206 (i.e., a cylindrical edge surface 304) may be preferred as suppressing posterior capsule opacification.

FIG. 3 further illustrates the function of the IOL 200 with respect to “near normal light” 306. The near-normal light 306 has an angle of incidence 308 with respect to the optical axis 204 such that the near-normal light 306 will be focused by the optic 202 onto the fovea. In some embodiments, near-normal light 306 may be defined as that which is focused by the optic 202 within the perimeter of the parafovea (a region of the retina surrounding the fovea) or within the perimeter of the perifovea (a region of the retina surrounding the parafovea). For example, near-normal light 306 may be defined as having an angle of incidence 308 of less than or equal to 2.5 degrees, i.e., the visual angle for light focused on the fovea. In other embodiments, near normal light 306 may be defined as having an angle of incidence 308 of less than or equal to 4.2 degrees (the visual angle of the parafovea) or less than or equal to 9.2 degrees (the visual angle of the perifovea) with respect to the optical axis 204.

It is desired that near normal light in the human visible spectrum be transmitted through the optic 202 and that the portion of a reflected portion 310 of the near-normal light 306 that falls within the human visible spectrum be reduced or eliminated. Reducing the reflected portion 310 of near-normal light 306 in the human visible spectrum improves the vision of the patient and reduces the appearance of “crazy eye.”

To that end, the anterior surface 300a within a radius 312 extending out from the optical axis 204 may have a metasurface formed thereon or adhered thereto that reduces reflection of light in the human visible spectrum. The radius 312 may extend completely or partially to the perimeter of the optic 202, such as to a radius of 2.25 to 3.75 mm. The radius 312 may coincide with the extent of refractive and/or diffractive structures formed on the anterior surface 300a and/or posterior surface 300b. In some exemplary embodiments, the metasurface is formed on or adhered to the anterior surface 300a, which may be planar or non-planar, whereas refractive and diffractive structures are defined on the posterior surface 300b, which may be planar or non-planar. In additional or alternative embodiments, the metasurface may be formed on or adhered to the anterior surface 300a, which may also comprise refractive and/or diffractive structures.

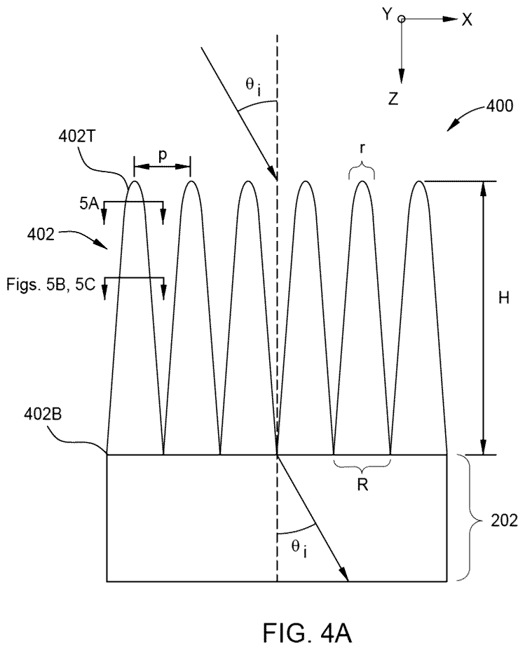

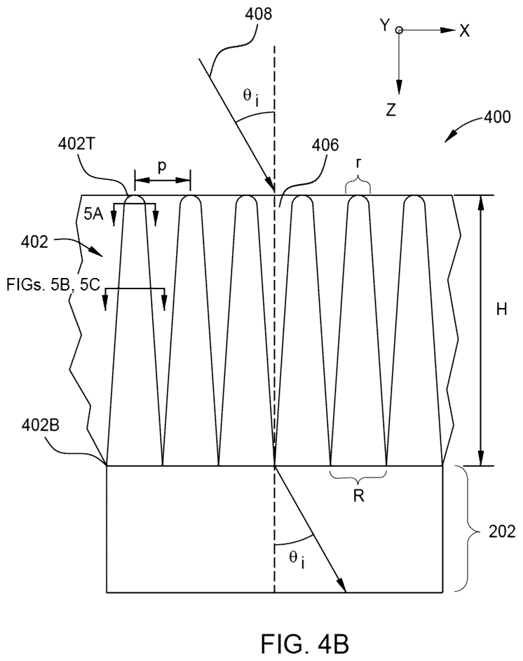

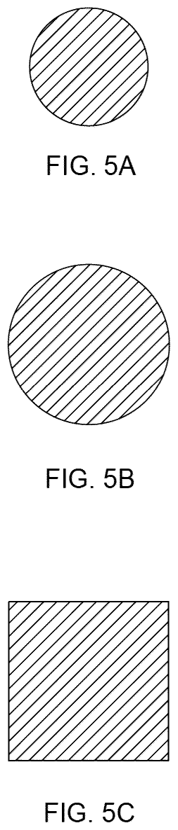

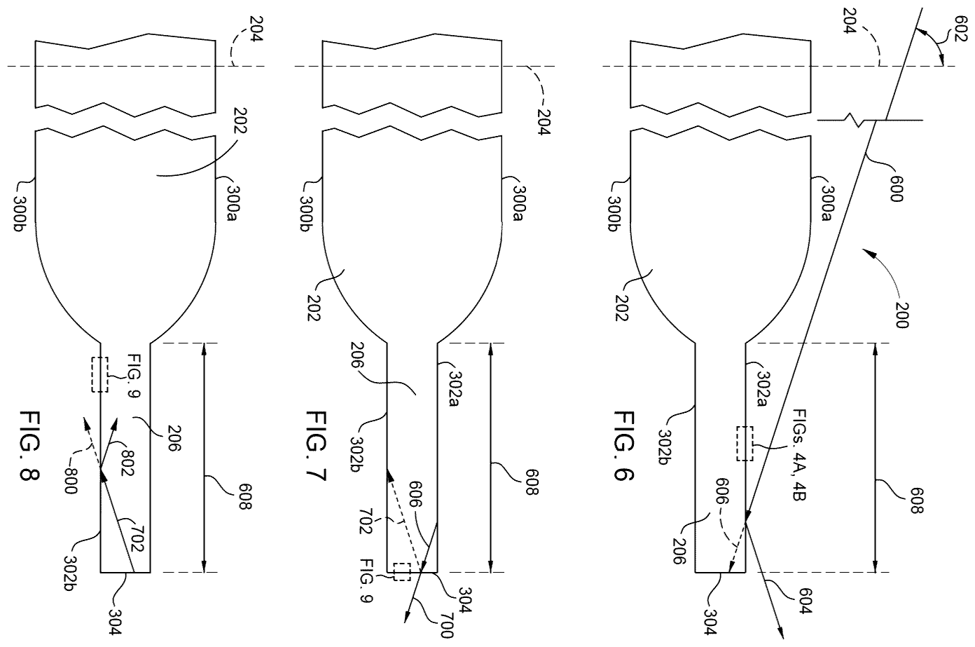

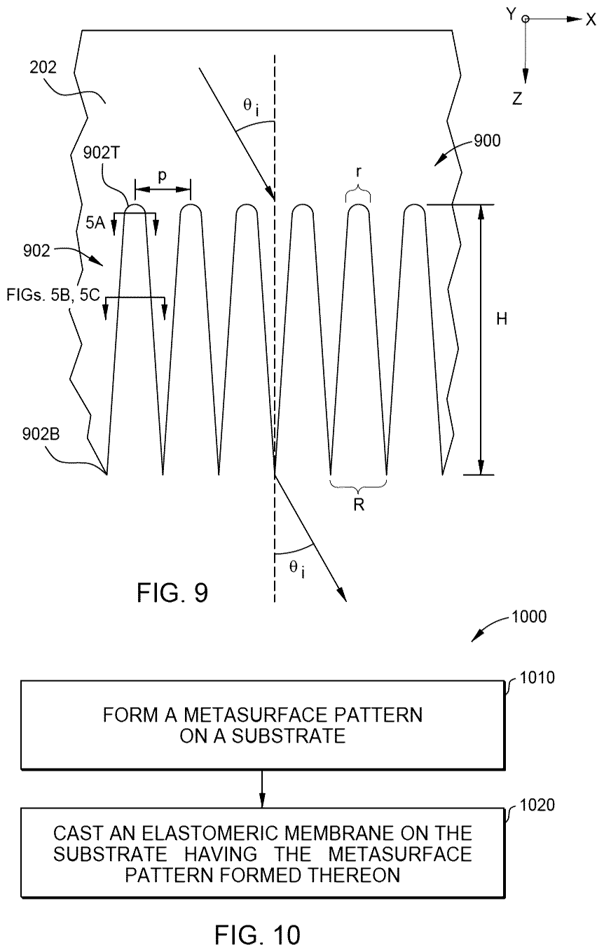

As shown in FIGS. 4A and 4B, a metasurface 400 may be formed within the radius 312 on the anterior surface 300a of the optic 202, and may comprise a plurality of pillars 402, which may be arranged on or formed in the anterior surface 300a of the optic 202. The pillars 402 may taper from a widened base 402B to a smaller top 402T. The pillars 402 may have a conical shape with a rounded tip having a radius of curvature r. The pillars 402 may have heights H of between about 30 nm and about 1000 nm in a Z direction defined as normal to the surface from which an individual pillar 402 extends, such as the anterior surface 300a of the optic 202 and possibly the anterior surface 302a of the peripheral portion 206 of the IOL 200. The pillars 402 may have widths R of between about 30 nm and about 500 nm in X and Y directions that are defined as perpendicular to the Z direction, i.e., tangent to a point on the surface from which an individual pillar 402 extends. The pillars 402 may have a pitch P between about 30 nm and about 500 nm in the X direction and in the Y direction. The heights H, widths R, and pitch P of the pillars 402 may be constant throughout the metasurface 400 or may vary, such as with distance from the optical axis 204. The pillars 402 may have a circular cross section from base 402B to top 402T (sec FIGS. 5A and 5B). Alternatively, the pillars 402 may transition from a square or rectangular cross section at the base 402B (see FIG. 5C) to a round cross section near the top 402T (e.g., within 0.05*H from the top 402T). The illustrated shape of the pillars 402 is exemplary only. The pillars 402 may alternatively be implemented as nano-fins (e.g., having a cross-section in the X-Y plane with an aspect ratio greater than 2, greater than 4, or greater than 8), nano-cones, or similar.

The pillars 402 may extend from the anterior surface 300a of the optic 202 into the aqueous humor within the eye of the patient as shown in FIG. 4A or may be embedded in a coating material 406 as shown in FIG. 4B. In either embodiment, the pillars 402 may be made of the same material as the bulk of the optic 202 or may be of a different material having a different index of refraction from the remainder of the optic 202. Where a coating material 406 is used, the coating material 406 may match more closely the index of refraction of the aqueous humor to reduce reflections. The coating material 406 may alternatively be formed of the same material forming the bulk of the optic 202 with the pillars 402 formed of a material having a contrasting index of refraction from the material forming the bulk of the optic 202.

Light 408 is incident on the metasurface 400 at an angle of incidence θᵢ. The reflectivity of the metasurface 400 with respect to the wavelength of incident light is a function of the height H, the width R, pitch P, angle of incidence θ₁, and the index of refraction of the pillars 402 relative to the surrounding medium (aqueous humor or coating material 406).

The properties of the pillars 402 (height H, the width R, pitch P, and, as far as they can be selected, index of refraction of the pillars 402 and the surrounding medium) may be selected such that the metasurface 400 acts as a long pass filter for near-normal angles of incidence θᵢ. The periodic arrangement and size of the pillars 402 may function to reflect light waves at different depths, creating wavelength-dependent constructive and destructive interference. Where reflected light from different depths interferes destructively at a given wavelength, transmission occurs at that wavelength without attenuation. Where reflected light from different depths interferes constructively at a given wavelength, reflection (i.e., attenuation of transmission) occurs at that wavelength. The degree of constructive and destructive interference determines the degree of attenuation of transmitted light at a given wavelength.

A long pass filter has a “cut on” wavelength such that wavelengths shorter than the cut on wavelength will be attenuated, such as by at least 3 dB, and wavelengths longer than the cut on wavelength will not be attenuated, e.g., attenuated by less than 3 dB. To reduce “crazy eye” the cut on wavelength may be selected to be just below the visible spectrum, such as 380 nm, 370 nm, or 360 nm. In this manner, light in the visible spectrum will be transmitted through the metasurface 400 rather than reflecting. Transmission of light with wavelengths shorter than the cut on wavelength, e.g., 380 nm, will be attenuated and therefore such wavelengths will be reflected. The cut on wavelength increases with increase in θᵢ. Accordingly, the properties of the pillars 402 may be selected to provide a cut on wavelength just below the visible spectrum for all near normal light, such as near-normal light 306 as defined above with respect to FIG. 3.

Accordingly, near-normal light 306 that is above the cut on wavelength (e.g., above 380 nm and therefore in the visible spectrum) will be transmitted without substantial (e.g., greater than 3 dB) attenuation and therefore without substantial reflection. Because the light does not substantially reflect, it is not visible to others as “crazy eye.” Transmission of light below the cut on wavelength (e.g., below 380 nm and therefore not in the visible spectrum) is attenuated. Light below the cut on wavelength will therefore be reflected. However, since light below the cut on wavelength is not in the visible spectrum, it is not perceptible to others and will not contribute to crazy eye.

Table 1, below, provides example values for metasurface 400, including a pillar height (H), pitch/period (P), cone tip radius of curvature (r), and pillar width (R); refractive index of material forming pillars 402 (nₙ) and the refractive index of the medium surrounding the pillars (nₐ).

TABLE 1

| | | Tip Radius | | nₙ (pillar | nₐ (medium |
|---|---|---|---|---|---|
| Height | Period | of Curvature | Width | refractive | refractive |
| (H) | (P), | (r) | (R) | index) | index) |
| 1000 nm | 280 nm | 17 nm | 250 nm | 1.5898 | 1.336 |

A further advantage provided by the pillars 402 may include an improved wetting property of an IOL such as superhydrophobicity. Superhydrophobicity may be achieved by dimensioning and arranging the pillars 402 with a height-to-pitch ratio (H/P) of between about or including 2.5 and 5, thereby providing a metasurface 400 with desired optical, wetting and antimicrobial properties. In one aspect, pillars 402 may be arranged to provide superhydrophilicity properties for the metasurface by having a height-to-pitch ratio of between about or including 1 and 2 and a pitch within the range of 0.1 μm to 1 μm or a pitch with an end value of said pitch range. The improved wetting from superhydrophilicity may reduce scattering of light by air bubbles trapped among the pillars 402. Antimicrobial properties may be the result of the pillars 402 reducing adhesion of bacteria as described in the following document, which is hereby incorporated herein by reference in its entirety: Kim S, Jung U T, Kim S K, Lee J H, Choi H S, Kim C S, Jeong M Y, “Nanostructured multifunctional surface with antireflective and antimicrobial characteristics,” ACS Appl Mater Interfaces (January 2015).

FIG. 6 illustrates the behavior of the IOL 200 with respect to “oblique light” 600. In the description of FIG. 6, as well as FIGS. 7 and 8, the oblique light 600, as well as portions thereof described as being transmitted or reflected from the surfaces described below, may be understood as light in the visible spectrum with behavior of light outside of the visible spectrum being ignored as not contributing to crazy eye or positive dysphotopsia. As used herein oblique light 600 may be defined as light having an angle of incidence 602 with respect to an optical axis, for example optical axis 204 that is larger than the largest angle of incidence 308 for near-normal light 306 as defined above with respect to FIG. 3. In some embodiments oblique light 600 may be defined as light having an angle of incidence 602 such that the light is incident on the peripheral portion 206 of the IOL 200. Such oblique light 600 may lead to undesirable visual disturbances, such as positive dysphotopsia, in the absence of some mitigating solution such as the metasurface 400 as described herein. For example, in some instances, such oblique light 600 may be defined as having an angle of incidence of at least 40 degrees, at least 50 degrees, at least 60 degrees, or at least 70 degrees.

When oblique light 600 is incident on the anterior surface 302a of the peripheral portion 206, a portion of the incident oblique light 600, reflected light 604, is reflected off of the anterior surface 302a of the peripheral portion 206, and a portion of the incident oblique light 600, transmitted portion 606, is transmitted into the peripheral portion 206. As the transmitted portion 606 travels internally through the peripheral portion 206, the transmitted portion 606 may then be incident on and reflected from the edge surface 304 of the peripheral portion 206. As previously discussed, light, such as transmitted portion 606, that reflects off of the edge of an IOL, such as edge surface 304, may then strike a portion of the retina, thereby causing undesirable positive dysphotopsia.

To mitigate the above-mentioned phenomenon of positive dysphotopsia caused by the transmitted portion 606 reflecting off of edge surface 304, the anterior surface 302a of the peripheral portion 206 of the IOL 200 may include a metasurface, such as metasurface 400, formed thereon to reduce the transmitted portion 606 within the visible spectrum. For example, the anterior and/or posterior surfaces 302a, 302b of the peripheral portion 206 may have a metasurface 400 formed thereon, such as within an annular region 608 of the peripheral portion 206 extending about the optical axis 204. The annular region 608 may extend from the edge surface 304 partially or completely to the optic 202, such as to the portion of the optic 202 that is defined by the radius 312 (see FIG. 3). In some embodiments, the entire anterior surface of the IOL 200, possibly with the exception of the haptics 210, may have a metasurface 400 formed thereon. The metasurface 400 formed within the radius 312 may have the same properties (H, P, R, r, nₙ as defined above) or different properties from the metasurface 400 within the annular region 608.

In some embodiments, the anterior surface 300a of the optic 202 and the anterior surface 302a of the peripheral portion 206 are planar and coplanar with one another, with the posterior surface 300b of the optic 202 defining refractive and/or diffractive structures. In some embodiments, a single manufacturing step may be used to form a metasurface 400 on the anterior surface 300a of the optic 202 and the anterior surface 302a of the peripheral portion 206.

As noted above, the cut on wavelength for the long pass filter defined by metasurface 400 increases with increase in the angle of incidence θᵢ. This property may advantageously be used to provide metasurface 400 that does not substantially reflect near-normal light 306 in the visible spectrum in order to reduce crazy eye while also reflecting oblique light 600 in the visible spectrum in order to reduce positive dysphotopsia. Stated differently, as the cut on wavelength is shifted higher with increasing angle of incidence θᵢ, eventually the human visible spectrum will fall below the cut on wavelength and transmitted light in the visible spectrum will be substantially, e.g., more than 3 dB, attenuated. Wavelengths longer than the cut on wavelength, such as near infrared and infrared light may still be transmitted but will not be visible and therefore will not contribute to positive dysphotopsia.

The angle of incidence of the near-normal light 306 is much smaller than the angle of incidence of the oblique light 600. The cut on wavelength increases with an increase in θᵢ. Accordingly, identically configured metasurfaces 400 (e.g., H, P, R, r, nₙ being identical within manufacturing tolerances and/or the metasurfaces being formed during the same manufacturing process) formed on the anterior surface 300a of the optic 202 and the anterior surface 302a of the peripheral portion 206 may achieve both a reduction in crazy eye and a reduction in positive dysphotopsia. For example, the metasurfaces 400 may be configured with a cut on wavelength below the visible spectrum (e.g., ˜380 nm) for near-normal light 306 and a cut on wavelength shifted to above the visible spectrum (e.g., ˜750 nm) for oblique light 600. Near-normal light 306 will therefore be transmitted rather than reflected, which reduces crazy eye. Oblique light 600 will be reflected rather than entering the IOL 200, thereby reducing internal reflection causing positive dysphotopsia.

For example, Table 2 illustrates, for a metasurface 400 configured according to Table 1, the variation in the cut on wavelength (λₘᵢₙ) for various oblique angles of incidence θᵢ. As is readily apparent, for angles of incidence greater than 60 degrees, the cut on wavelength λₘᵢₙ is longer than the longest visible wavelength (˜750 nm). Therefore the metasurface 400 acting as a longpass filter will substantially block transmission of all visible light.

TABLE 2

| θᵢ (deg) | λₘᵢₙ (nm) |
|---|---|
| 40 | 693 |
| 50 | 739 |
| 60 | 778 |
| 70 | 805 |

Referring to FIG. 7, the transmitted portion 606 of oblique light 600 that is incident on the edge surface 304 includes a transmitted portion 700 and a reflected portion 702. The reflected portion 702 causes positive dysphotopsia and therefore may be reduced. For example, a metasurface 400 may be formed on or secured to the edge surface 304 and have properties (H, P, R, r, nₙ as defined above) selected to transmit wavelengths in the visible spectrum for possible angles of incidence for the transmitted portion 606. The metasurface formed on or secured to the edge surface 304 may be configured as either (a) a longpass filter with the cut on wavelength shorter than the human visible spectrum (e.g., less than 380 nm) or (b) a shortpass filter with a cut off wavelength that is longer than the visible spectrum (e.g., greater than 750 nm). The cut off wavelength of a short pass filter is the wavelength above which transmission of wavelengths will be substantially, e.g., at least 3 dB, attenuated (e.g., reflected) and below which transmission of wavelengths will not be substantially attenuated.

Note that in some embodiments, a metasurface on the anterior surface 302a of the peripheral portion 206 is sufficient such that a metasurface on the edge surface 304 is not present. In other embodiments, a metasurface is formed on the edge surface 304 and omitted from the anterior surface 302a.

Referring to FIG. 8, the reflected portion 702 may then be incident on the posterior surface 302b of the peripheral portion 206 resulting in a transmitted portion 800 and a reflected portion 802. The transmitted portion 800 will cause positive dysphotopsia upon incidence on the retina. It is therefore desired to reduce the transmitted portion 800.

The same principle could be applied to Blue light filtering. Under near-normal incident light on IOL (θᵢ=0 degree) cut on wavelength (λmin) passing through IOL could be achieved by configuring the pillars to filter blue light. An example pillar configuration for blue light filtering (2 min=400 nm) is provided below in Table 3.

TABLE 3

| | | Tip Radius | | nₙ (pillar | nₐ (medium |
|---|---|---|---|---|---|
| Height | Period | of | Width | refractive | refractive |
| (H) | (P), | Curvature (r) | (R) | index) | index) |
| 1000 nm | 250 nm | 17 nm | 200 nm | 1.5898 | 1.336 |

In some embodiments, a metasurface may be formed all, or a portion of, the posterior surface 302b, such as within the same annular region 608 or a different annular region 608. The metasurface formed on the posterior surface 302b may be configured as either (a) a longpass filter with the cut on wavelength longer than the visible spectrum (e.g., greater than 750 nm) or (b) a shortpass filter with a cut off wavelength that is shorter than the visible spectrum (e.g., less than 380 nm).

Note that in some embodiments, a metasurface on the anterior surface 302a of the peripheral portion 206 and/or edge surface 304 is sufficient such that a metasurface on the posterior surface 302b is not present. In still other embodiments, a metasurface on the posterior surface 302b is used alone and metasurfaces on the anterior surface 302a of the peripheral portion 206 and/or edge surface 304 are omitted.

Referring to FIG. 9, for the scenarios illustrated in FIGS. 7 and 8, light is transmitted through a high-index medium (the IOL 200) to an interface with a low index medium (the aqueous humor, vitreous humor, or capsular bag) as the light exits the IOL 200, which is the opposite from the scenarios of FIGS. 3 and 6. The metasurface formed on or secured to the edge surface 304 and/or the posterior surface 302b may therefore have a different configuration from the metasurface 400 that may be positioned on the anterior surface 300a of the optic 202.

In particular, a metasurface 900 formed on or secured to the edge surface 304 and/or the posterior surface 302b may include indentations 902 rather than pillars 402, the indentations having a height H, width R, tip radius r, and pitch P. The cross section of each indentation may be round from a bottom 902B (i.e., open end) of the indentation 902 to a top 902T (i.e., deepest point) of the indentation 902 (see FIGS. 5A and 5B). Alternatively, the indentations 902 may transition from a square or rectangular cross section at the bottom 902B (see FIG. 5C) to a round cross section near the top 902T (e.g., within 0.05*H from the top 902T). The values for H, R, r, and P for the metasurface 900 may be selected in the same manner as corresponding values for the metasurface 400 and may have the same ranges of possible values as listed above. The indentations 902 may be filled with fluid (e.g., the vitreous) during use or may be filled with a coating material as described above with respect to FIG. 5B.

In one aspect, a metasurface, such as metasurface 400 or 900, is formed on a substrate by standard micro/nano fabrication methods. FIG. 10 depicts an example method 1000 for forming a metasurface of an IOL 200. Block 1010 includes forming a metasurface pattern on a substrate. For example, a photolithography process may be performed to spin on a photoresist on the substrate, and selected areas of the photoresist are exposed to light and developed. Then, the pattern of the photoresist is transferred to the substrate by reactive ion etching. Thereafter, the remaining photoresist is removed by plasma over-etching.

The metasurface pattern may extend over an area of about 7 mm by about 7 mm on the substrate, and may include an array of trenches carved in the substrate with heights of between about 30 nm and about 1000 nm, widths of between about 30 nm and about 500 nm, and spacing between adjacent trenches of between about 30 nm and about 500 nm.

The term “substrate” as used herein refers to a layer of material that serves as a basis for subsequent processing operations and includes a surface to be cleaned. For example, the substrate may include glass, or one or more conductive metals, such as nickel, titanium, platinum, molybdenum, rhenium, osmium, chromium, iron, aluminum, copper, tungsten, or combinations thereof.

The substrate can also include one or more materials comprising silicon, including materials associated with group IV or group III-V including compounds, such as Si, polysilicon, amorphous silicon, silicon nitride, silicon oxynitride, silicon oxide, Ge, SiGe, GaAs, InP, InAs, GaAs, GaP, InGaAs, InGaAsP, GaSb, InSb and the like, or combinations thereof. Furthermore, the substrate can also include dielectric materials such as silicon dioxide, organosilicates, and carbon doped silicon oxides. Further, the substrate can include any other materials such as metal nitrides, metal oxides and metal alloys, depending on the application.

Moreover, the substrate is not limited to any particular size or shape. The substrate can be a round wafer having a 200 mm diameter, a 300 mm diameter, a 450 mm diameter or other diameters. The substrate can also be any polygonal, square, rectangular, curved or otherwise non-circular workpiece, such as a polygonal glass, plastic substrate.

Block 1020 includes casting an elastomeric membrane on the substrate that has the metasurface pattern formed thereon. The elastomeric membrane may be formed of a thin hydrophobic acrylic polymeric material. In the casting process, a hydrophobic acrylic polymeric material in a liquid form may be mixed with a cross-linking agent and poured onto the substrate and then heated at an elevated temperature, thereby hardening and cross-linking the hydrophobic acrylic polymeric material, to form the elastomeric membrane. The elastomeric membrane may have a thickness of between about 200 μm and 500 μm and replicates the metasurface of the substrate. The metasurface elements may have heights of between about 30 nm and about 200 nm, widths of between about 30 nm and about 300 nm, and spacings between adjacent trenches of between about 30 nm and about 300 nm. After casting, the elastomeric membrane is removed from the substrate. This elastomeric membrane can be used as an anterior surface, a posterior surface, and/or optic edge of an IOL. Additionally or alternatively, the elastomeric membrane may be an embedded within an IOL.

Other possible manufacturing methods include additive and/or subtractive manufacturing of metasurface elements (e.g., 3D printing), imprinting metasurface elements, tuned diamond turning, and/or lithography techniques that operate directly onto an elastomeric surface for providing metasurface elements.

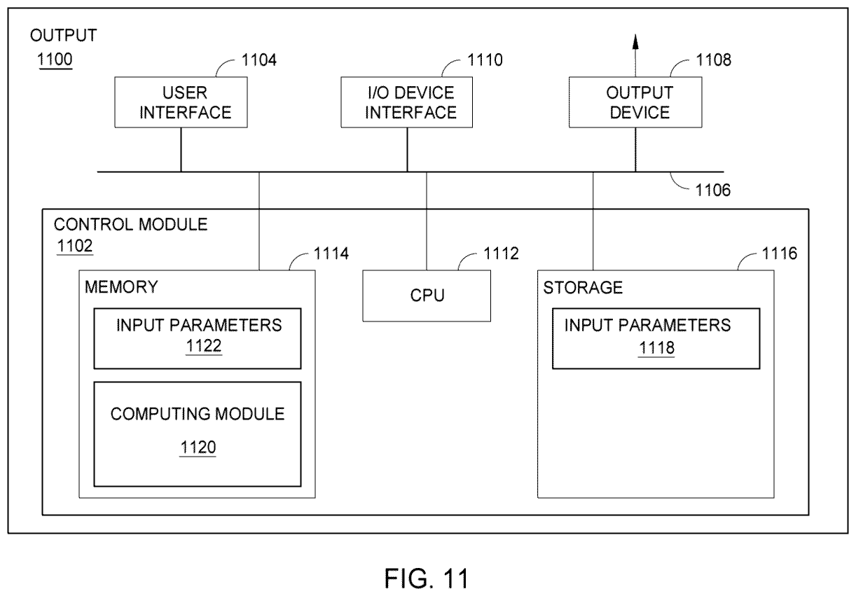

FIG. 11 depicts an exemplary system 1100 for designing, configuring, and/or forming an IOL 200 according to any of the embodiments described above. As shown, the system 1100 includes, without limitation, a control module 1102, a user interface display 1104, an interconnect 1106, an output device 1108, and at least one I/O device interface 1110, which may allow for the connection of various I/O devices (e.g., keyboards, displays, mouse devices, pen input, etc.) to the system 1100.

The control module 1102 includes a central processing unit (CPU) 1112, a memory 1114, and a storage 1116. The CPU 1112 may retrieve and execute programming instructions stored in the memory 1114. Similarly, the CPU 1112 may retrieve and store application data residing in the memory 1114. The interconnect 1106 transmits programming instructions and application data, among CPU 1112, the I/O device interface 1110, the user interface display 1104, the memory 1114, the storage 1116, output device 1108, etc.

The CPU 1112 can represent a single CPU, multiple CPUs, a single CPU having multiple processing cores, and the like. Additionally, in certain embodiments, the memory 1114 represents volatile memory, such as random-access memory. Furthermore, in certain embodiments, the storage 1116 may be non-volatile memory, such as a disk drive, solid state drive, or a collection of storage devices distributed across multiple storage systems.

As shown, the storage 1116 includes input parameters 1118. The input parameters 1118 may include a lens base power, a desired value of refractive index of an optic, and/or an optical longpass filter cut-on wavelength. The memory 1114 includes a computing module 1120 for computing control parameters, such as the arrangement of metasurface elements of metasurface 400, 900 (e.g., pillar shape, size, and pitch or density). In addition, the memory 1114 includes input parameters 1122.

In certain embodiments, input parameters 1122 correspond to input parameters 1118 or at least a subset thereof. In such embodiments, during the computation of the control parameters, the input parameters 1122 are retrieved from the storage 1116 and executed in the memory 1114. In such an example, the computing module 1120 comprises executable instructions for computing the control parameters, based on the input parameters 1122. In certain other embodiments, input parameters 1122 correspond to parameters received from a user through user interface display 1104. In such embodiments, the computing module 1120 comprises executable instructions for computing the control parameters, based on information received from the user interface display 1104.

In certain embodiments, the computed control parameters, are output via the output device 1108 to a lens manufacturing system that is configured to receive the control parameters and form a lens accordingly. In certain other embodiments, the system 1100 itself is representative of at least a part of a lens manufacturing systems. In such embodiments, the control module 1102 then causes hardware components (not shown) of system 1100 to form the lens according to the control parameters by the operations described above.

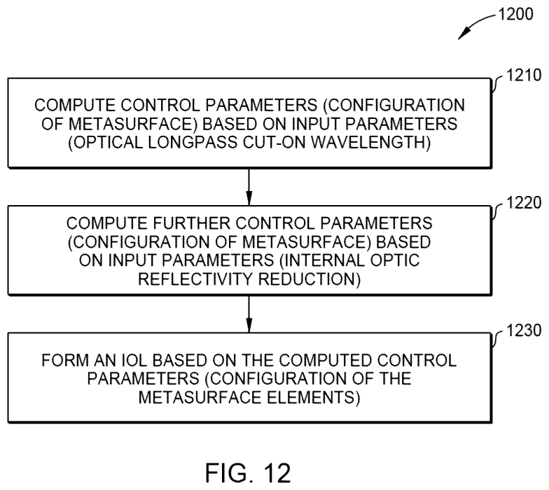

FIG. 12 depicts example operations 1200 for forming an IOL (e.g., IOL 200). In some embodiments, the steps 1210 and 1220 of operations 1200 are performed by one system (e.g., the system 1100) while step 1230 is performed by a lens manufacturing system. In some other embodiments, steps 1210, 1220, and 1230 are performed by a lens manufacturing system.

At step 1210, control parameters, such as configuration of the metasurface 400, 900 (e.g., pillar shapes, sizes, and density/pitch) are computed based on input parameters (e.g., an optical longpass cut-on wavelength).

At step 1220, further control parameters, such as configuration of the metasurface 400, 900 (e.g., pillar shapes, sizes, and density/pitch) are computed based on input parameters (e.g., a chosen spectrum of light and degree of reduction thereof of internal optic reflections).

At step 1230, an IOL (e.g., IOL 200) based on the computed control parameters, such as configuration of the metasurfaces 400, 900 (e.g., pillar shapes, sizes, and density/pitch), may be formed according to the operations described above, using appropriate methods, systems, and devices typically used for manufacturing lenses, as known to one of ordinary skill in the art.

While the foregoing is directed to embodiments of the present disclosure, other and further embodiments of the disclosure may be devised without departing from the basic scope thereof. and the scope thereof is determined by the claims that follow.

1. An intraocular lens (IOL) comprising:
an optic having an anterior optic surface configured to receive light passing into an eye in which the IOL is implanted and a posterior optic surface opposite the anterior optic surface, the IOL including at least one of refractive and diffractive structures configured to improve vision of the eye; and
a metasurface configured to reduce reflection in a human visible spectrum from the anterior optic surface.

2. The IOL of claim 1, wherein the metasurface is at least one of formed on and secured to the anterior optic surface.

3. The IOL of claim 1, wherein the metasurface comprises an optical longpass filter with a cut on wavelength below the human visible spectrum.

4. The IOL of claim 1, wherein the optic defines an optical axis and the metasurface comprises an optical longpass filter with a cut on wavelength below the human visible spectrum for angles of incidence relative to the optical axis of less than 9.2 degrees.

5. The IOL of claim 4, wherein the cut on wavelength is smaller than 380 nm.

6. The IOL of claim 1, wherein the metasurface comprises pillars.

7. The IOL of claim 6, wherein the pillars comprise tapered pillars.

8. The IOL of claim 7, wherein the tapered pillars comprise monotonically tapered pillars.

9. The IOL of claim 6, wherein the pillars are arranged with a pillar height-to-pillar pitch ratio of about 2.5 to 5.

10. The IOL of claim 6, wherein the pillars have heights of between 30 nm and 1000 nm, widths of between 30 nm and 500 nm, and pillar pitch of between 30 nm and 500 nm.

11. The IOL of claim 1, further comprising one or more haptics that extend from the optic.

12. The IOL of claim 1, further comprising a peripheral portion extending around the optic, the peripheral portion including an edge surface configured to engage a capsular bag of the eye.

13. The IOL of claim 12, wherein:
the metasurface is a first metasurface;
the peripheral portion includes an anterior peripheral surface and a posterior peripheral surface opposite the anterior peripheral surface, the anterior peripheral surface configured receive the light passing into the eye; and
a second metasurface is at least one of formed on or secured to the peripheral portion, the second metasurface configured to increase reflection in a human visible spectrum from the anterior peripheral surface.

14. The IOL of claim 13, wherein the second metasurface comprises pillars.

15. The IOL of claim 13, wherein the second metasurface is configured to increase reflection in the human visible spectrum from the anterior peripheral surface for angles of incidence greater than 60 degrees.

16. The IOL of claim 14, wherein the first metasurface is configured to reduce reflection in the human visible spectrum from the anterior peripheral surface for angles of incidence less than 9.2 degrees.

17. The IOL of claim 16, wherein the second metasurface comprises an optical longpass filter with a cut on wavelength below the human visible spectrum for angles of incidence less than 9.2 degrees and a cut on wavelength above the human visible spectrum for angles of incidence greater than 60 degrees.

18. The IOL of claim 13, further comprising a cylindrical edge surface extending around the peripheral portion and positioned between the anterior peripheral surface and the posterior peripheral surface.

19. The IOL of claim 18, further comprising a third metasurface at least one of formed on or secured to the cylindrical edge surface, the metasurface configured to reduce reflection into the optic from the cylindrical edge surface.

20. The IOL of claim 19, wherein the third metasurface comprises an array of tapered indentations extending into the posterior peripheral surface.
